(19) United States
(12) Patent Application Publication
(10) Pub. No.: US 2013/0303947 A1
(43) Pub. Date: Nov. 14, 2013

(54) SPASTICITY MEASURING DEVICE

(71) Applicants: Didier Gamet, Compiegne Cedex (FR); Kevin Buffenoir-Billet, Nantes (FR); Chantal Perot, Compiegne Cedex (FR)

(72) Inventors: Didier Gamet, Compiegne Cedex (FR); Kevin Buffenoir-Billet, Nantes (FR); Chantal Perot, Compiegne Cedex (FR)

(21) Appl. No.: 13/980,034

(22) PCT Filed: Jan. 17, 2012
(86) PCT No.: PCT/EP12/50649

§ 371 Date: Jul. 16, 2013

(30) Foreign Application Priority Data
Jan. 17, 2011 (FR) 1150361

(51) Int. Cl. A61B 5/11; A61B 5/0492

(52) U.S. Cl. CPC A61B 5/1107; A61B 5/0492
USPC 600/595; 600/546

(57) ABSTRACT

The invention relates to a device for measuring the spasticity of an articulation of an individual, the articulation being located between a first limb and a second limb, said device being characterised in that it comprises: a first part for receiving all or part of the first limb of the articulation; a second part for receiving all or part of the second limb of the same articulation, the second part being pivotably connected to the first part about a rotational axis; and means for determining a spasticity score from measurements relating to the reaction of said articulation to a flexion exerted on the articulation, the determination means comprising a means for measuring an articulation reaction torque at the site of the rotational axis.

The invention relates to the medical field and more specifically to the field of measuring spasticity.

It will be recalled that spasticity is an exaggeration of the myotatic reflex. More specifically, it is an exaggerated reflex contraction of a muscle in reaction to its stretching. Spasticity can be uniform over the whole body but it is most usually localised on the lower limbs (spastic diplegia) or on one side of the body.

To determine in a non-invasive manner the presence of spasticity and to be able to evaluate it there exists a procedure known as the “Ashworth manoeuvre” (Ashworth B., Preliminary trial of carisoprodol in multiple sclerosis, Practitioner 1964; 192:540-542), a manoeuvre which characterises the degree of spasticity. This manoeuvre consists in bending passively the articulation of the ankle of the patient, which triggers in the spastic individual a stretching reflex characterised by a brief extension of the articulation. This is perceived by the practitioner and makes it possible to qualify the extent of the spasticity on a specific scale known as the “Modified Ashworth Scale” ranging from 0 to 4:
0: Normal muscle tone,
1: Slight increase in the muscle tone, manifested by a catch followed by a release or by minimal resistance at the end of the range of motion,
1+: Slight increase in muscle tone, manifested by a catch, followed by minimal resistance perceived over less than half of the range of motion,
2: More marked increase in muscle tone through most of the range of motion, but the articulation can be easily moved,
3: Considerable increase in muscle tone, making passive movement difficult.
4: The affected articulation is rigid in flexion or extension (abduction or adduction).

This technique thus enables an evaluation of spasticity. Nevertheless, this manoeuvre involves the tactile assessment of the practitioner; it thus remains purely qualitative and highly subjective.

In order to enable a more objective measurement, myometers enabling muscle tone to be measured are known. Nevertheless, numerous criticisms have been made against these devices in that they do not make it possible, in themselves, to measure spasticity; in fact, this is not linked uniquely to muscle tone. Moreover, these devices are difficult to handle during the Ashworth manoeuvre since the latter requires both hands of the practitioner.

International application WO 2010/121353 describes a portable device for measuring spasticity, which is based on a Canadian theory that describes spasticity by means of the occurrence of electromyographic activity and the speed of movement from a remarkable angle known as “Dynamic Stretch Reflex Threshold”.

This device thus makes it possible to identify spasticity impairment in a patient, but in no case to describe it on the Ashworth scale.

Consequently, an aim of the present invention is to provide a device for measuring the spasticity of an individual making it possible to determine a spasticity score in an objective manner in order to refine in a substantial manner the diagnosis of impairment.

Another aim of the present invention to provide a device for measuring spasticity that can be handled easily by the practitioner.

In this respect, the invention relates to a device for measuring the spasticity of an articulation of an individual, the articulation being located between a first limb and a second limb, the device being characterised in that it comprises:
a first part for receiving all or part of the first limb of the articulation,
a second part for receiving all or part of the second limb of the articulation, the second part being pivotably connected to the first part about a rotational axis, and
means for determining a spasticity score from measurements relating to the reaction of said articulation to a flexion exerted on the articulation.

Advantageously, but optionally, the invention comprises at least one of the following characteristics:
the determination means comprise a means for measuring an articulation reaction torque at the site of the rotational axis,
the determination means further comprise a unit for processing the measurement of the articulation reaction torque at the site of the rotational axis by the measuring means, the processing unit being intended to determine:
   the continuous component of said measurement,
   the alternating component of said measurement,
the determination means comprise a means for measuring the muscular activity of part of the muscles of the articulation,
the means for measuring the muscular activity of part of the muscles of the articulation comprises at least two electrodes making it possible to establish an electromyogram of said muscular activity,
the distance between the at least two electrodes and the rotational axis is adjustable,
the determination means comprise a means for measuring the angle formed between the two parts of the device,
at least one of the first and second parts of the device comprises an upper surface and the distance between said upper surface and the rotational axis is adjustable,
the determination means further comprise data transmission means, preferably wireless transmission means.

The invention relates to a device for measuring the spasticity of an ankle of an individual, the ankle being located between a foot and the lower part of a leg of the individual, the device being characterised in that it comprises:
a first part for receiving all or part of the sole of the foot of the individual,
a second part for receiving all or part of the second limb of the articulation of the individual, the second part being pivotably connected to the first part about a rotational axis, and
means for determining a spasticity score from measurements monitoring the reaction of the ankle to a flexion exerted thereon:
   the continuous component of the reaction torque of the ankle at the site of the rotational axis,
   the alternating component of the reaction torque of the ankle at the site of the rotational axis,
   the muscular activity of the extensor muscles,
   the angle formed between the two parts of the device.

The invention relates to a method of using a device for measuring the spasticity of an articulation of an individual located between a first limb and a second limb, the method being characterised in that it comprises at least the following steps:
providing a device according to the invention,
placing the first limb of the articulation at the level of the first part,
placing the second limb at the level of the second part,
applying a flexion to the articulation,
measuring the reaction of said articulation,
determining a spasticity score.

Other characteristics, aims and advantages of the present invention will become clear on reading the detailed description that follows of a non-limiting implementation example, given with reference to the appended figures, amongst which:

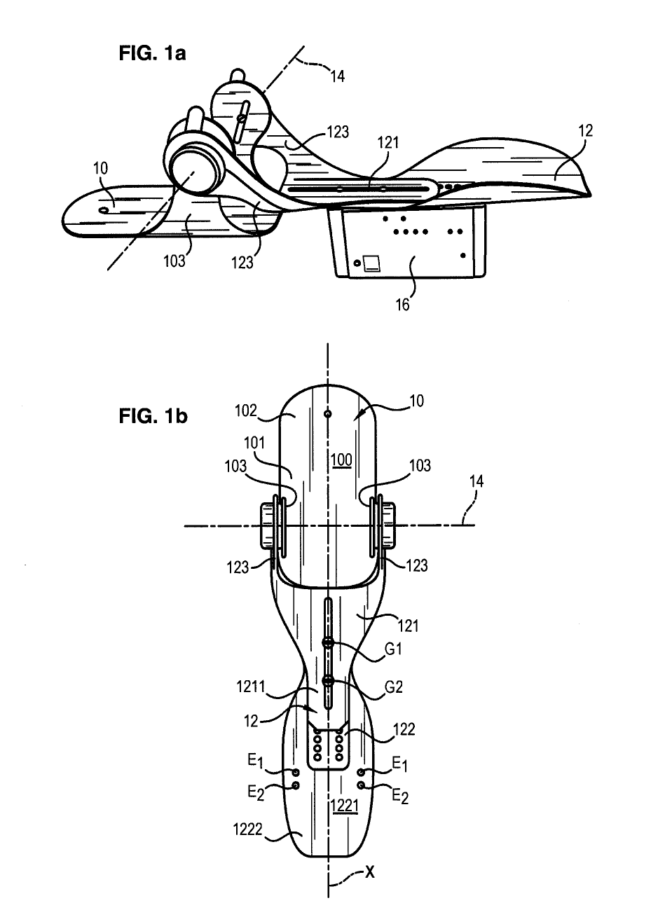

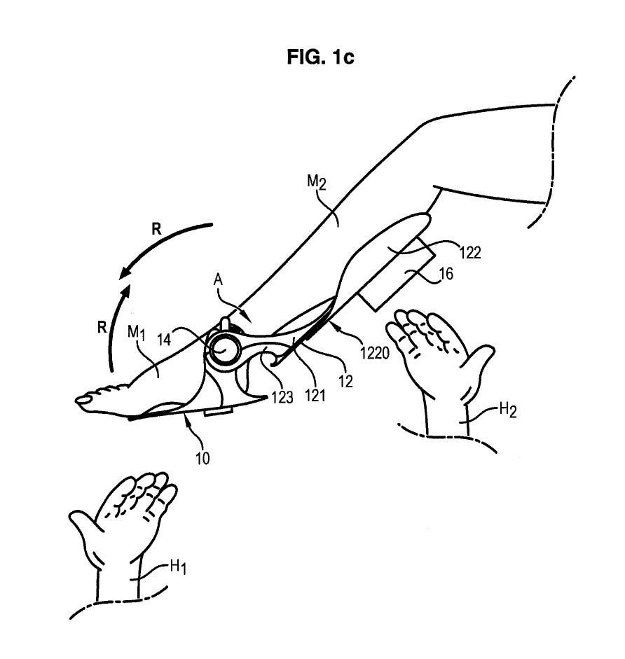

FIG. 1a is a cavalier view representation of the device according to a particular embodiment of the present invention,

FIG. 1b is a schematic top view representation of the device according to a particular embodiment of the present invention,

FIG. 1e is a schematic side view representation of the device according to a particular embodiment of the present invention receiving an articulation of an individual,

FIG. 2 is a schematic side view representation of the pivot connection of the device according to a particular embodiment of the present invention,

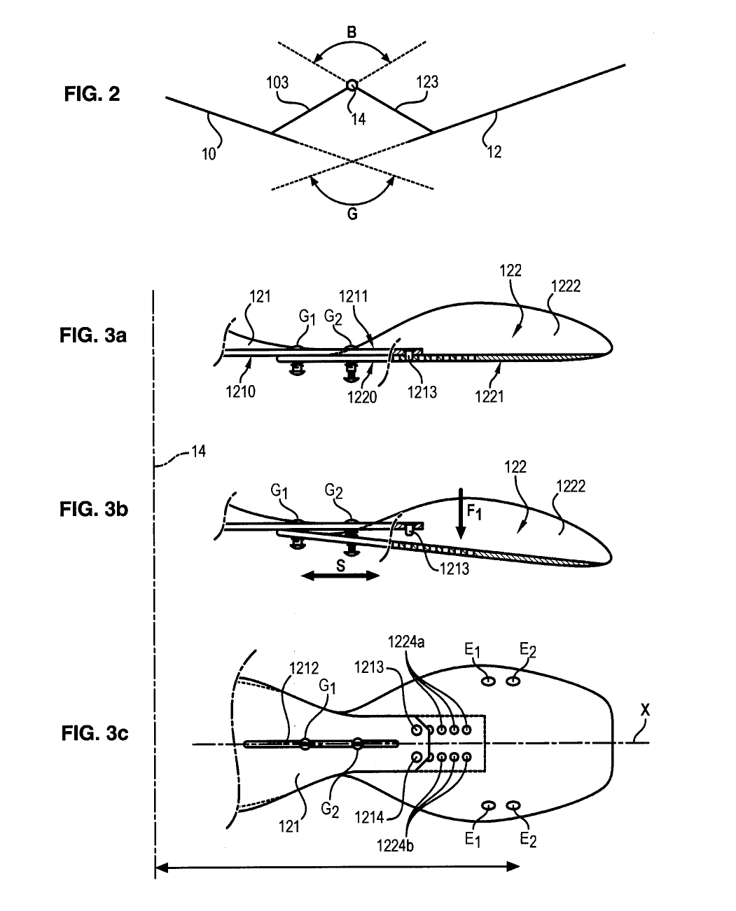

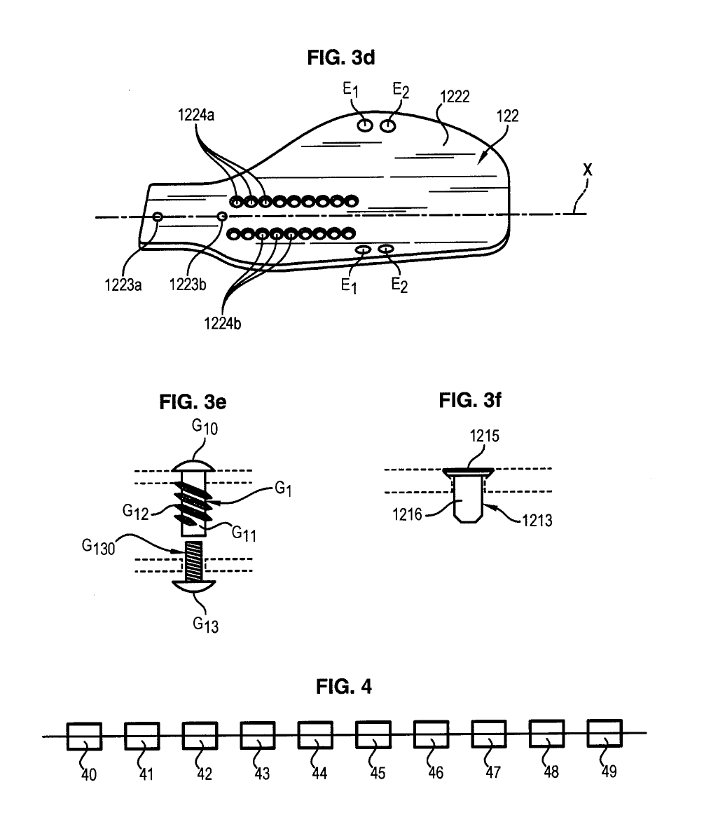

FIG. 3a is a schematic side view representation of the second part of the device according to a particular embodiment of the present invention,

FIG. 3b is another schematic side view representation of the second part of the device according to a particular embodiment of the present invention,

FIG. 3c is another schematic top view representation of the second part of the device according to a particular embodiment of the present invention,

FIG. 3d is a schematic cavalier view representation of the extension of the second part of the device according to a particular embodiment of the present invention,

FIG. 3e is an exploded schematic side view representation of a connecting element between the plate element and the extension of the second part of the device according to a particular embodiment of the present invention,

FIG. 3f is an exploded schematic side view representation of another connecting element between the plate element and the extension of the second part of the device according to a particular embodiment of the present invention,

FIG. 4 is a functional schematic graph representing a method of using the device according to the invention,

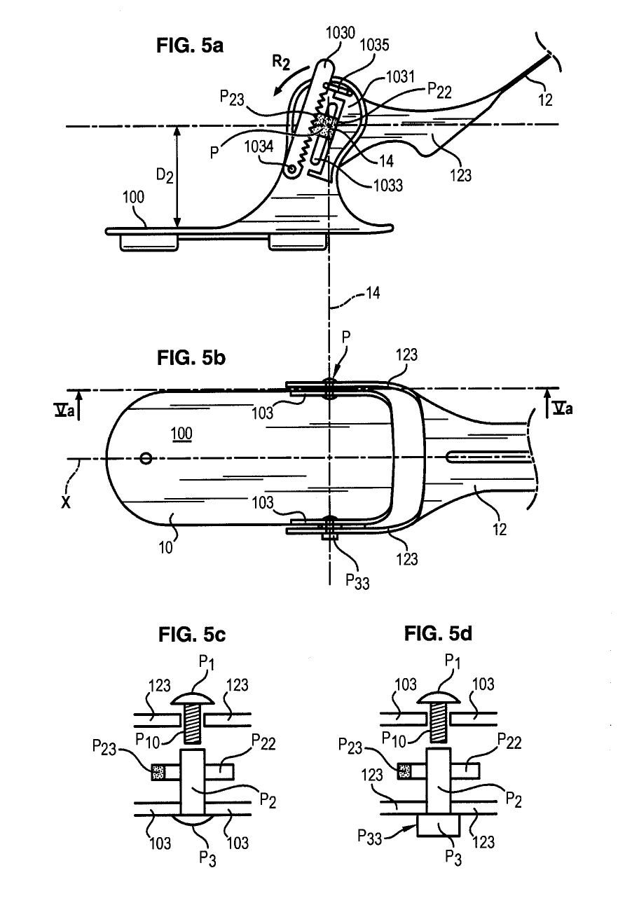

FIG. 5a is a side view of the device according to a particular embodiment of the present invention,

FIG. 5b is a schematic top view representation of the device according to a particular embodiment of the present invention,

FIG. 5c is an exploded side schematic representation of a connecting element between the first and the second part of the device according to a particular embodiment of the present invention,

FIG. 5d is an exploded side schematic representation of a connecting element between the first and the second part of the device according to a particular embodiment of the present invention, incorporating an angular sensor,

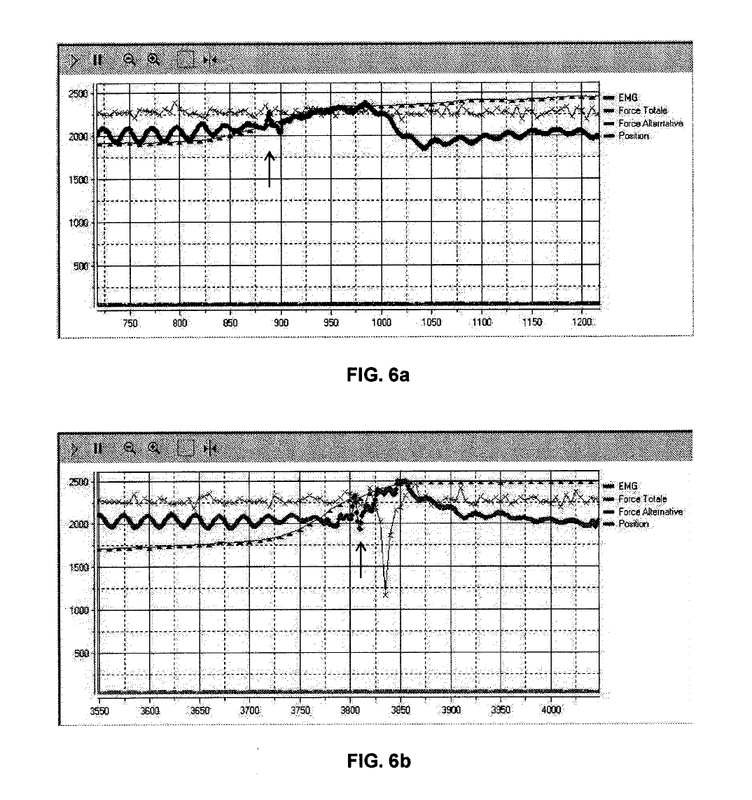

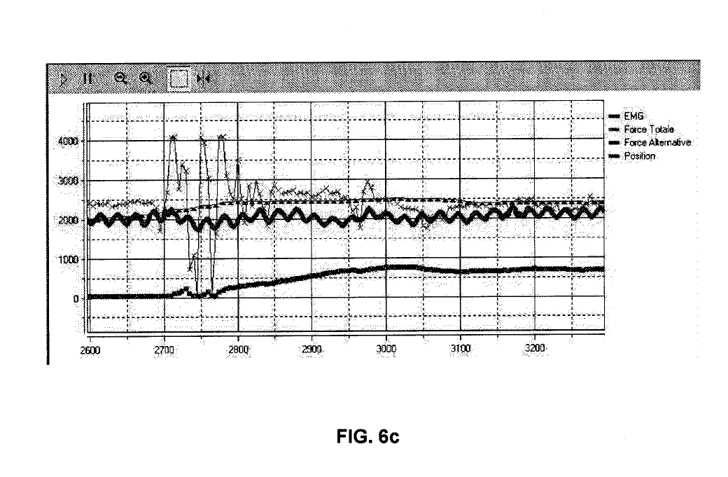

FIG. 6a represents the unprocessed signals obtained for a first patient during a slow manoeuvre test,

FIG. 6b represents the unprocessed signals obtained for a first patient during a rapid manoeuvre test,

FIG. 6c represents the unprocessed signals obtained for a second patient during a test,

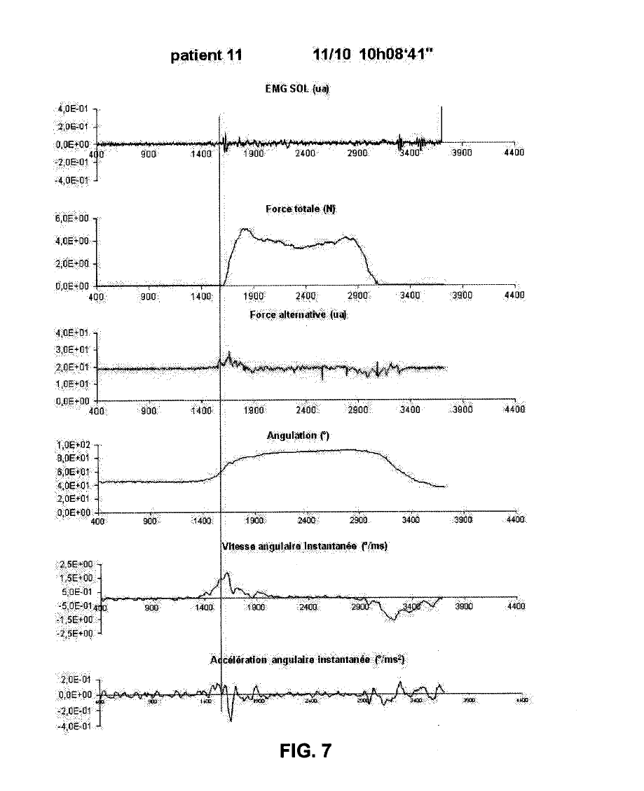

FIG. 7 represents the processed signals obtained for a third patient during a test.

With reference to FIGS. 1a to 1e, a device for measuring the spasticity of an articulation A, such as an ankle of an individual, located between a first limb M1 (such as the foot) and a second limb M2 (such as the lower part of the leg) comprises:
a first part 10 for receiving all or part of the first limb M1 of the articulation A of the individual,
a second part 12 for receiving all or part of the second limb M2 of the articulation A of the individual, the second part 12 being pivotably connected to the first part 10 about a rotational axis 14.

It should be noted that in the remainder of the text, “pivot connection” between the first and the second part must be understood in a wide sense. Although it is described herein as a pivot connection along a single axis, it may be easily generalised to several axes of rotation and thus become a ball and socket connection between the first and the second limb.

Preferentially, the first part 10 is a plate having an upper surface 100 for receiving the first limb M1. The first part 10 has a disc shape 102 with a rectangular extension 101 (extending in the same plane as the disc). The first part 10 comprises a hinge member 103 extending at the level of the rectangular extension 101 perpendicularly to the surface 100.

The second part 12, for its part, comprises a plate 121 having an upper surface 1211 for receiving a lower part of the limb M2 and a lower surface 1210. The plate 121 is overall rectangular, the edges being able to be rounded. The second part 12 comprises a hinge member 123 extending perpendicularly to the upper surface 1211.

The second part 12 also comprises an extension 122. Said extension 122 has a plate shape with an upper surface 1221 for receiving an upper part of the limb M2 and a lower surface 1220. The extension 122 and the plate 121 are connected such that the lower surface 1210 of the plate 121 and the upper surface 1221 of the extension 122 are parallel and in contact with each other. The connection between these two elements will be described in more detail hereafter. The extension 122 also comprises a rim 1222 on the circumference of one end of the extension 122. Said rim defines a concave wall towards a point situated above the surface 1221 such that said rim 1222 makes it possible to form a housing intended to receive an upper part of the second limb M2 and more particularly the calf in the case of an ankle.

The hinge members 103 and 123 (respectively of the first part 10 and of the second part) are connected to each other such that the second part 12 is pivotably connected to the first part 10 about a rotational axis 14. This connection will be described in greater detail hereafter.

The device also comprises means for determining 16 a spasticity score from measurements relating to the reaction of the articulation A to a flexion exerted on the articulation A of the individual, flexion exerted by the practitioner. The method of using the device will be described in detail hereafter. The determination means 16 may be located on the first or on the second part of the device. Alternatively, the determination means may be in the form of several interconnected parts, each situated on a part of the device.

The determination means 16 comprise a means for measuring 160 (not represented) a reaction torque (exerted by the articulation A in reaction to the flexion) at the site of the rotational axis 14. In the case of an ankle, this measuring means makes it possible to determine the pressing force of the sole of the foot in reaction to the flexion exerted on the ankle. In fact, a torque, or “moment of force” is in mechanics a rotational stress applied to an axis (here the axis 14) under the effect of a force (in this instance the pressing force of the sole of the foot). The moment in relation to a point O of a force F, the application point of which is at the point M is defined by:

[symbol]

ₒ=O\vec{M} [symbol] \vec{F}(M)

In the case of an ankle, the measuring means 160 may consist either of a torque meter performing a measurement of the torque in N·m⁻¹ at the point of the axis 14 (from which is deducted the value of the pressing force), or an active force sensor (as an example, the XFL225D FGP Sensors®) placed in the first part 10 which measures directly the pressing force of the sole of the foot.

This force parameter, which was never measured by known devices, makes it possible to bring the Ashworth qualitative scale closer to the shape (amplitude and time) of the signals obtained.

The determination means further comprise a means for processing 161 (not represented) a signal emitted by the means for measuring 160 the reaction torque of the articulation A at the site of the rotational axis 14, the processing unit 161 being intended to determine:
the continuous component of said signal,
the alternating component of said signal.

Such a determination of the continuous and alternating components of the signal is easily accessible by those skilled in the art, for example by means of filtering elements widely known from the prior art. As an example, it is possible to implement a high pass filter at 3 Hz with an amplification of a factor 50 to determine the alternating component.

The determination means 16 comprise a means for measuring 162 (not represented) the muscular activity of at least one part of the muscles of the articulation A. More specifically, the means for measuring 162 the muscular activity of part of the muscles of the articulation A comprises at least two electrodes E1 and E2, preferentially laid out at the site of the rim 1222 of the extension 122 making it possible to establish an electromyogram of the muscular activity of the upper part of the limb M2. In the case of an ankle, the measuring means 162 make it possible to establish an electromyogram of the extensor muscles of the ankle such as the soleus muscle and/or the gastrocnemius muscles. The establishment of such an electromyogram from electrodes is widely known in the prior art.

With reference to FIG. 2, the determination means 16 also comprise a means for measuring 163 (not represented) the angle G between the first part 10 and the second part 12. Nevertheless, the only measurement that is accessible directly is the measurement of the angle P formed between the two hinge members 103 and 123 of the device at the site of the rotational axis 14. With reference to FIG. 2 schematically representing the device, the hinge member 103 has an angle α₁ with the surface 100 of the first part 10. The hinge member 123 has an angle α₂ with the surface 1211 of the second part 12. Consequently, in order to obtain the value of the angle G that the surfaces 100 and 1211 have (respectively of the first 10 and the second 12 part) as a function of the angle β measured at the site of the rotational axis 14, it suffices to apply the following relation:

G=α₁+α₂−β

α₁ and α₂ being fixed, a simple taring suffices to determine the angle G from the measurement of the angle β and vice versa, β being the image of G to within a constant.

Thus the determination means 16 collect the following measured information:
the continuous component of the articulation A reaction torque at the site of the rotational axis 14,
the alternating component of the articulation A reaction torque at the site of the rotational axis 14,
the muscular activity of part of the muscles of the articulation A,
the angle G formed between the two parts 10 and 12 of the device at the site of the rotational axis 14.

From this information, the determination means 16 determine a spasticity score.

The calculation of a score is preferentially based on a normalisation over time of the manoeuvre, on an integration of the force signal, a rating according to the angulation and the speed. Preferentially, the overall score is determined by a linear combination of the four items of information.

Advantageously, the determination means 16 comprise a calculation unit (such as a microcontroller) and a data transmission means 164 (not represented), preferentially a wireless transmission means, for example by Wi-Fi or Bluetooth protocol, to a remote reception device (not represented) such as a computer, thereby making it possible to save the information measured and/or the spasticity score.

Alternatively, the spasticity score is determined by the remote receiving device from the transmitted measured information. Advantageously, the score is displayed by the computer or by a display device located on the device, such as an LCD screen, intended for the practitioner (the display being of sufficient size so that the practitioner can easily read the information displayed). Alternatively, the score is transmitted to the practitioner in the form of a sound (diction of the score, emission of a more or less high-pitched or loud tonality depending on the score) emitted by a loudspeaker element arranged on the device. From this score, the practitioner can establish a diagnosis concerning the spasticity of the individual and optionally propose a treatment to administer.

Preferentially the device has a symmetry along its longitudinal axis X. Thus, a same device is just as suitable for a left articulation as a right articulation. In addition, the device may be handled by a right handed or left handed practitioner, elements for transmitting the score to the practitioner being provided on both sides of the device or being moveable so as to be visible from both sides of the device.

With reference to FIGS. 3a to 3d, the plate 121 comprises an upper face 1211 for receiving a lower part of the limb M2 and a lower surface 1210 intended to be in contact with the extension 122. The plate 121 comprises, on its face 1211, an oblong through slot 1212 arranged on the median along the main axis X of the plate 121. On the lower surface 1210, at the level of an end of the plate 121, are arranged two protruding lugs 1213 and 1214.

The extension 122 comprises a face 1221 for receiving an upper part of the limb M2 and to be in contact with the lower face 1210 of the plate 121. The extension 122 comprises two principal through holes 1223a and 1223b arranged on the median along the main axis X of the extension 122 such that once the extension 122 and the plate 121 are matched, the hand holes 1223a and 1223b are aligned vertically with the slot 1212 of the plate 121. The extension 122 also comprises two series of secondary through holes 1224a and 1224b situated on either side of the median along the main axis X of the extension 122. Each series of through holes is aligned parallel to the main axis X of the extension 122.

The plate 121 and the extension 122 are connected such that the hand holes 1223a and 1223b of the extension 122 are aligned vertically with the slot 1212 of the plate 121. Two connecting elements G1 and G2 are inserted in the principal through holes 1223a and 1223b in order to serve as guiding elements and thus to assure a sliding connection between the plate 121 and the extension 122 along the main axis X.

With reference to FIG. 3e, each connecting element G1 and G2 comprises an upper abutment G10 intended to come into contact with the upper surface 1211 of the plate 121, the abutment G10 being preferentially cylindrical of circular section of a diameter greater than the width of the slot 1212 in order to be able to serve as abutment. Each connecting element G1 and G2 also comprises a rod G11 connected to the abutment element G10, the diameter of said rod being less than the width of the slot 1212 and the diameter of the hand holes 1223a and 1223b. Thus, the rod passes through the slot 1210 and the hand holes 1223a and 1223b. Each connecting element G1 and G2 also comprises a resilient element such as a spring G12 associated with a lower abutment G13. Preferentially, this lower abutment G13 may be separated from the remainder of the connecting element and is fixed by screwing a threaded part G130 of the lower abutment G13 in a hole tapped in the rod G11. The resilient element is intended to be in contact with the lower surface 1220 of the extension 122 such that the connecting elements G1 and G2 exert a pressure between the plate 121 and the extension 122 in the sense of placing in contact the surface 1210 of the plate 121 and the surface 1221 of the extension 122.

With reference to FIG. 3f, the lug 1213 (similar to the lug 1214) comprises an abutment part 1215 intended to come into contact with the surface 1211 of the plate 121 and a body 1216 intended to be housed in one of the holes of the two series of secondary through holes 1224a and 1224b. Preferentially, the lug 1213 is fixed by tightening in a tower of the plate 121 provided to receive the lug.

Once the two surfaces 1210 and 1221 have been placed in contact, the lugs 1214 and 1213 (represented transparently in FIG. 3c) are respectively inserted into one of the holes of the two series of secondary through holes 1224a and 1224b thereby making it possible to block in translation the plate 121 and the extension 122.

Thus the distance D between the two electrodes E1 and E2 and the rotational axis 14 is adjustable. In fact, to modify the distance D, the practitioner exerts a pressure Fi on the extension 122 against the pressure exerted by the connecting elements G1 and G2 in order to free the lugs 1213 and 1214 from the secondary through holes 1224a and 1224b as represented in FIG. 3b. Once the lugs have been freed, the sliding connection between the plate 121 and the extension 122, assured by the connecting elements G1 and G2, is freed and the practitioner (while maintaining the pressure Fi) can modify the distance D by manoeuvring the extension 122 along the direction S along its main axis. Once the distance has been chosen, the practitioner releases the pressure Fi that he was exerting and the pressure of the connecting elements G1 and G2 enables the plate 121 and the extension 122 to be replaced in contact and an insertion of the lugs 1213 and 1234 respectively in one of the holes of the series of secondary through holes 1224a and 1224b thereby making it possible to block in translation the plate 121 and the extension 122 (as represented in FIG. 3a).

Such a layout makes it possible to place correctly the electrodes E1 and E2 with respect to the limb M2 of the articulation A. The practitioner places correctly the electrodes at the level of the muscle(s) to receive at the second part 12 of the device and thus guarantee an electromyogram of good quality of the muscular activity of the limb M2.

With reference to FIGS. 5a and 5b, the distance D₂ between the surface 100 of the first part 10 of the device and the axis 14 is adjustable. To this end, the hinge member 103 of the first part 10 comprises a slot 1033 extending transversally on the surface 100 of the first part 10 and preferentially perpendicularly to the axis 14. A rack element 1030 is pivotably fixed about a pivot element 1034 (and parallel to the axis 14) to the hinge member 103 such that the toothing of the rack element 1030 is parallel to the through slot 1033. The hinge member 123 of the part 12 is connected to the hinge member 103 by a connecting element P.

With reference to FIG. 5c, the connecting element P comprises an abutment P1 and a rod P2, the diameter of the rod being of diameter less than the width of the slot 1033 and a through hole of the hinge member 123 of the part 12 into which the connecting element P is inserted. The connecting element P comprises a stop element P3. Thus the abutments P1 and P3 are intended to come into contact with the hinge members 103 and 123. The rod P2 of the connecting element P further comprises a central part P22 comprising a toothed part P23 intended to come into cooperation with the rack element 1030. In order to maintain the toothed part P23 cooperating with the rack element 1030, a maintaining element 1031 is provided, making it possible to maintain the central part P22 in locked position. Preferentially the rack element 1030, the maintaining element 1031 and the central part P22 are all three situated between the hinge member 103 and the hinge member 123. The maintaining is for example achieved by a pressure exerted by a return spring 1035 connected between the rack element 1030 and the hinge member 103 making it possible to exert a pressure on the rack element 1030 in the direction of the maintaining element 1031. Once in locked position, the toothing P23 is maintained in position by the rack element 1030 and the connecting element P is consequently maintained in position in the slot 1030. The connecting element P being pivotably connected to the hinge member 123 of the second part 12, once the assembly has been locked, the second part 12 is no longer only pivotably connected to the first part 10. When the assembly is unlocked (by freeing the rack element 1030 going against the force of the return spring 1035, along the direction R2), the connecting element P is in free circulation along the slot 1030.

Thus, when the practitioner wishes to adjust the distance D2, he unlocks the rack element 1030, which makes it possible to free the toothed part P23. The connecting element P is thus again free to slide along the slot 1033. Once the correct position has been chosen, the practitioner locks the rack element 1030 which maintains the connecting element P in position in the slot 1033 by means of the return spring exerting a pressure on the rack element. Consequently, the practitioner can modify the distance D2 separating the surface 100 from the rotational axis 14 in order to adapt the device to the articulation A that it has to receive.

Preferentially, the upper abutment PI can be separated from the remainder of the connecting element P and is fixed by screwing of a threaded part P10 of the upper abutment PI in a hole tapped within the rod P2.

Preferentially, the connection as described is made in two symmetrical parts laid out on either side of the main axis X as represented in FIG. 5b. With reference to FIG. 5e (and as also represented in FIG. 5b), at least one of the two connecting elements P comprises an angular sensor P33 situated in abutment and making it possible to measure the angular position of one hinge member compared to the other (as explained previously).

With reference to FIG. 4, the invention also relates to a method of using the device of the invention comprising the following steps:
providing a device according to the invention (step 40). For example the practitioner (doctor, physiotherapist, nurse, nursing auxiliary, etc.) can have such a device available in his surgery,
then, the practitioner places the articulation of the individual whose spasticity he wishes to measure. To do this, he places the first limb of the articulation, for example the foot, at the level of the first part of the device (step 41),
he also places the second limb of the same articulation, for example the lower part of the leg, at the level of the second part of the device (step 42),
the practitioner then applies a flexion to the articulation along the direction R represented in FIG. 1c (step 43). To this end, the practitioner takes in one hand H1 the first part 10 and in the other hand H2 the second part 12 to operate a flexion of the articulation in the direction R, as represented in FIG. 1e,
the reaction of said articulation A is then measured (step 44) as explained previously. To this end, it is for example provided that the practitioner triggers the acquisition of signals by pressing on a button fixed on the device, for example on a part housing the acquisition means 16. Alternatively, the acquisition is triggered automatically as soon as the device reaches a predetermined angle between its first and its second part,
the information is optionally sent by wireless transmission to a data receiving device such as a computer to save the data and/or to display the spasticity score (step 45),
the practitioner waits for the stoppage of the acquisitions (it is possible for example to select beforehand the duration of the acquisitions). Alternatively, the practitioner stops the acquisition by pressing on a button provided for this purpose, which may be the same button as for the start of acquisition (step 46),
determination of the spasticity score from the measurements carried out as explained previously (step 47),
display of the spasticity score (step 48),
from the information at his disposal, the practitioner makes a diagnosis concerning the spasticity of the individual and an optional treatment to administer (step 49).

It should be noted that the device according to the invention has been the subject of two series of evaluations on spastic patients, at Saint-Jacques hospital in Nantes.

For the first evaluation series, four spastic patients participated in the orthosis tests. These patients are affected by spasticity to various degrees, from 1+ to 3, on the modified Ashworth scale. For each patient, the device was tested after different clinical tests (of an average duration of an hour) concerning their spasticity at the level of the lower limbs such as Qualified Walking Analysis, tendon and muscular stimulations (soleus muscle) and ankle movement exercises.

The articular and muscular solicitation tends to cause an adaptation of the stretched muscles. The more the stretching reflex has been solicited, the more the individual will react rapidly to an Ashworth manipulation. Thus, within the scope of the experimentation, it should be noted that the four patients tested were in the same context because they performed the same exercises beforehand.

For the second evaluation series, two patients participated in the test; the first, with the same conditions as those of the first series, whereas the other patient was evaluated before the clinical tests.

The experiments were performed with the knee braced. No particular instruction was given to the practitioner concerning the handling of the orthosis.

The soleus muscle was chosen for the measurement of the electrical activity (EMG).

As result, during the manoeuvres performed, the practitioners expressed no trouble and were able to score the patients. The feeling with or without the orthosis gives the same score for the same patient.

The results obtained on the patients tested are in the form of graphic results (FIGS. 6a-c) and numerical results.

In graphic form, the temporal variations of the signals acquired show that the patients tested have a spastic impairment either through the occurrence of a single catch or through the occurrence of a clonus.

The Ashworth manoeuvre is carried out at a dorsiflexion speed called slow or rapid by the practitioner. Slow speed designates an instantaneous speed estimated between 0.05 and 0.09°/ms (n=11) and rapid speed designates an instantaneous speed estimated between 0.12 and 0.6°/ms (n=9). FIGS. 6a and 6b represent the unprocessed signals obtained for Patient 2 (impairment level 1+) during a slow (FIG. 6a) and rapid (FIG. 6b) manoeuvre.

The 4 unprocessed signals (figure a) are the total force (FT), the alternating force (FA), the angular position (Angulation or Position) and the electromyogram of the soleus muscle.

During the dorsiflexion test, the force catch is visible on the alternating component of the force (FA) as being the variable part of the total force (FT) but also on the EMG signal of the soleus (see arrow).

Given the different starting angulation of the ankle, depending on whether the test is carried out at slow or rapid speed, the catch is triggered for a different angular position.

The low level of the spasticity of a Patient (1+) is in agreement with the low level of reaction force during dorsiflexion (FIG. 6a); the opposite is true as shown in FIG. 6c obtained for Patient 4, who has a spasticity level of 3 on the Ashworth scale which comprises 5 levels.

The triggering of a clonus is noted, which is exhaustible and a passive reaction of the patient that is more important than for the previous patient.

The processing software makes it possible to calculate the angular speed and acceleration from the angular position signal and also to filter the signals.

The results can be exported to a spread sheet programme (Excel® type) and make it possible to obtain the lines, such as those of patient 11 (impairment level 1) which are represented in FIG. 7

The catch or the start of a clonus are identifiable at the level of the alternating force signals (FA) or/and the EMG of the soleus. After the visual determination of the catch, the angulation values of the ankle, the average and instantaneous speed and instantaneous acceleration are noted and reported in a table.

As an example, for Patient 4 mentioned previously (who had an impairment level of 3) the following table is obtained:

| | AMS 3 | | | Angular | | Average | Positions |
|---|---|---|---|---|---|---|---|
| pa- | EMG | FT | FA | position | Speed | speed | Min Max |
| tient 4 | (au) | (N) | (au) | (°) | (°/ms) | (°/ms) | (°) |
| Test 1 | 0.014 | 0.041 | 18.01 | 61.58 | 0.103 | 0.077 | 54.2-27.6 |
| T = | | | | | | | |
| 2204 | | | | | | | |
| Test 2 | −0.003 | 0.547 | 19.01 | 67.28 | 0.173 | 0.047 | 62.2-97 |
| T = | | | | | | | |
| 1647 | | | | | | | |

To exploit these values an algorithm is then proposed using the “signal” parameters leading to the elaboration of a scale equivalent to that of Ashworth, but also adaptable to other known scales.

According to the modified Ashworth scale,
Normal muscle tone

May be translated by no EMG activity, a signal of position, speed and acceleration by the practitioner but at the end of the dorsiflexion movement a slight increase of the pressing force or muscular “tonus” is noted as resulting from the stretching of the group of extensors, the appearance of which is comparable to the stretching of a spring.
Slight increase in the muscle tone manifested by a catch followed by a release or by a minimal resistance at the end of movement

This level is identified by a catch at the end of movement accompanied by an increase in the muscle tone i.e. a pressing force greater than that of level 0.

1+Slight increase in the muscle tone manifested by a catch followed by a minimal resistance perceived over less than half of the range of motion

This level is comparable to the preceding level but the resistance or pressing force is perceived over less than half of the range of motion, i.e. after half the angulation up to the end of the movement.

2 More marked increase in the muscle tone affecting the major part of the range of motion, the articulation being able to be moved easily

This level is comparable to the previous level but the resistance or pressing force is perceived during dorsiflexion. However the pressing force on the whole of the dorsiflexion remains weak.

3 Important increase in the muscle tone making passive mobilisation difficult

This level is comparable to the previous level but the resistance or pressing force is important during dorsiflexion.

4 The articulation concerned is rigid in flexion or in extension, abduction or adduction

This level indicates a difficulty in mobilising the articulation or a very strong pressing force. In this case, the variation in the angulation is small for a strong pressing force.

According to the Held-Tardieu scale,

0 No resistance throughout the passive movement

This level is comparable to level 0 of the Ashworth scale.

1 Slight increase in resistance during passive movement without a catch at a precise angle being able to be clearly felt

This level is identified as being an intermediate level between level 0 and level 1 of the Ashworth scale. Only a slight increase in the force is noted, without triggering the catch.

2 Clear catch interrupting the passive movement at a precise angle, following a release

For this level, the catch is felt during the movement at a given angulation.

3 Exhaustible clonus (<10 s when the stretching is maintained) occurring at a precise angle

At this level, it is triggered at a given angulation, a trembling or clonus of a duration less than 10 s.

4 Inexhaustible clonus (>10 s when the stretching is maintained) occurring at a precise angle

This level is equivalent to the previous level but the clonus is inexhaustible, in other words it has a duration above 10 s.

According to the scale of rating stretching reflexes (Buffenoir et al.);

### 0 Absent

No catch, at slow or rapid speed.

### 1 Catch

Existence of a catch at a given angulation, at slow or rapid speed

2 Exhaustible clonus

Triggering of a clonus of short duration at a given angulation, at slow or rapid speed.

3 Inexhaustible clonus

Triggering of a clonus of long duration at a given angulation, at rapid speed.

4 Inexhaustible clonus at slow speed

Triggering of a clonus of long duration at a given angulation, at slow speed.

By virtue of the precise quantitative nature given by the different signals, it is proposed to group together these scales so as to obtain an overall scale with 25 levels.

This is based on the normalisations of the time and amplitude angulation signal and noting the occurrence of the increase in the reaction force.

The catch is identified on the EMG signal by a brief mark (stretch reflex) physiologically preceding the mechanical activity. Following the stretch reflex, a force opposing the pressing force is generated by the individual.

This force is weak compared to the pressing force or total force, but is noted on the alternating force as a force pulse; it is felt by the practitioner as being a catch by the patient. It is then possible to date it on the angulation signal.

1. Device for measuring the spasticity of an articulation (A) of an individual, the articulation being located between a first limb (M1) and a second limb (M2), said device being characterised in that it comprises:
a first part (10) for receiving all or part of the first limb (M1) of the articulation (A),
a second part (12) for receiving all or part of the second limb (M2) of the articulation (A), the second part (12) being pivotably connected to the first part (10) about a rotational axis (14), and
means for determining (16) a spasticity score from measurements relating to the reaction of said articulation (A) to a flexion exerted on the articulation (A), the determination means (16) comprising a means for measuring (160) an articulation (A) reaction torque at the site of the rotational axis (14).

2. Device according to claim 1, characterised in that the determination means (16) moreover comprise a unit for processing (161) the measurement of the articulation (A) reaction torque at the site of the rotational axis (14) by the measuring means (160), the processing unit (161) being intended to determine:
the continuous component of said measurement,
the alternating component of said measurement.

3. Device according to one of claims 1 to 2, characterised in that the determination means (16) comprise a means for measuring (162) the muscular activity of part of the muscles of the articulation (A).

4. Measuring device according to claim 3, characterised in that the means for measuring (162) the muscular activity of part of the muscles of the articulation (A) comprises at least two electrodes (E1, E2) making it possible to establish an electromyogram of said muscular activity.

5. Measuring device according to claim 4, characterised in that the distance (D) between the at least two electrodes (E1, E2) and the rotational axis (14) is adjustable.

6. Device according to one of claims 1 to 5, characterised in that the determination means (16) comprise a means for measuring the angle formed between the two parts (10, 12) of the device.

7. Device according to one of claims 1 to 6, characterised in that at least one of the first and second parts (10) of the device comprises an upper surface (100) and in that the distance (D2) between said upper surface (100) and the rotational axis (14) is adjustable.

8. Device according to one of claims 1 to 7, characterised in that the determination means (16) further comprises a data transmission means, preferentially a wireless transmission means.

9. Device for measuring the spasticity of an ankle (A) of an individual, the ankle being located between a foot (M1) and the lower part of a leg of the individual (M2), the device being characterised in that it comprises:
a first part (10) for receiving all or part of the sole of the foot (M1) of the individual (S),
a second part (12) for receiving all or part of the second limb (M2) of the articulation (A) of the individual (S), the second part (12) being pivotably connected to the first part (10) about a rotational axis (14), and
means for determining (16) a spasticity score from measurements following the reaction of the ankle (A) to a flexion exerted thereupon:
the continuous component of the reaction torque of the ankle (A) at the site of the rotational axis (14),
the alternating component of the reaction torque of the ankle (A) at the site of the rotational axis (14),
the muscular activity of the extensor muscles of the ankle such as the soleus muscle and/or the gastrocnemius muscles,
the angle (G) formed between the two parts (10, 12) of the device.

10. Method of using a device for measuring the spasticity of an articulation (A) of an individual located between a first limb (M1) and a second limb (M2), the method being characterised in that it comprises at least the following steps:
providing (40) a device according to one of claims 1 to 9,
placing (41) the first limb at the level of the first part,
placing (42) the second limb at the level of the second part,
applying (43) a flexion to the articulation (A),
measuring (44) the reaction of said articulation (A),
determining (47) a spasticity score.
